(19) United States
(12) Patent Application Publication
(10) Pub. No.: US 2024/0000871 A1
(43) Pub. Date: Jan. 4, 2024

(54) Lactobacillus gasseri capable of alleviating and treating hyperuricemia

(71) Applicant: Jiangnan University, Wuxi (CN)

(72) Inventors: Gang WANG, Wuxi (CN); Caixin NI, Wuxi (CN); Linlin WANG, Wuxi (CN); Jianxin ZHAO, Wuxi (CN); Hao ZHANG, Wuxi (CN); Wei CHEN, Wuxi (CN)

(21) Appl. No.: 18/335,275

(22) Filed: Jun. 15, 2023

Related U.S. Application Data
(63) Continuation of application No. PCT/CN2021/138680, filed on Dec. 16, 2021.

(30) Foreign Application Priority Data
Dec. 16, 2020 (CN) 2020114934939

(51) Int. Cl. A61K 35/747; C12N 1/20; A61P 19/06

(52) U.S. Cl. CPC A61K 35/747; C12N 1/205; A61P 19/06; C12R 2001/225

(57) ABSTRACT

The present disclosure discloses Lactobacillus gasseri capable of alleviating and treating hyperuricemia, which belongs to the technical field of microorganisms. The L. gasseri CCFM1133 of the present disclosure can reduce serum uric acid levels and the activity of serum and liver xanthine oxidase (XOD) of mice with hyperuricemia, and reduce the occurrence of hyperuricemia and gout; regulate glucose and serum triglyceride (TG) levels of patients with hyperuricemia, increase the activity of liver catalase (CAT) and glutathione peroxidase (GSH-Px); increase the expression of ileum ABCG2, promote the excretion of intestinal uric acid; and increase intestinal short-chain fatty acid levels to promote health. The L. gasseri CCFM1133 of the present disclosure can be used for preparing food, functional food or medicines, and has a wide application prospect.

### REFERENCE TO SEQUENCE LISTING

The instant application contains a Sequence Listing in XML format as a file named “YGHY-2023-17 SEQ.xml”, created on Jun. 12, 2023, of 5 kB in size, and which is hereby incorporated by reference in its entirety.

### TECHNICAL FIELD

The present disclosure relates to Lactobacillus gasseri capable of alleviating and treating hyperuricemia, which belongs to the technical field of microorganisms.

### BACKGROUND

Hyperuricemia (HUA) is a disease in which the uric acid level in the blood exceeds the normal value. In recent years, with the improvement of living standards, the incidence of hyperuricemia is also increasing. Patients with hyperuricemia account for about 13.3% of the total population in China. Uratoma caused by chronic hyperuricemia will further induce gout. Meanwhile, hyperuricemia is considered as a risk factor for cardiovascular and cerebrovascular diseases, chronic kidney diseases and atherosclerosis, which seriously threatens human health. Therefore, the treatment of hyperuricemia has attracted great attention. At present, the medicines for treating hyperuricemia mainly include allopurinol (a xanthine oxidase inhibitor), benzbromarone (a uricosuric medicine), and the like. However, these medicines have some side effects, and there are many controversies about the treatment of uric acid lowering medicine for asymptomatic hyperuricemia on the international. Therefore, diet and lifestyle improvement is a preferred method for treating asymptomatic hyperuricemia.

In recent years, with the in-depth research on the relationship between intestinal flora and human health, many studies have confirmed that probiotics can improve human health by regulating intestinal flora. The onset of hyperuricemia is closely related to the structural disorder of the intestinal flora, and the consumption of probiotics can regulate the intestinal microbiota by means of proliferating lactobacillus and bifidobacterium in intestinal tracts, improve the intestinal barrier function, reduce entrance of endotoxin and other metabolites into the liver along with the blood, and effectively reduce the blood uric acid level. Metabolite short-chain fatty acids of the intestinal flora play a key role in the regulation of host metabolism, immune systems and cell proliferation. Studies have shown that the intervention of sodium acetate can reduce serum uric acid and inhibit the activity of xanthine oxidase. The excretion of uric acid in the human body mainly depends on the excretion of kidney and intestinal tracts, ABCG2 as a uric acid transporter plays an important role in the intestinal excretion of uric acid, and the expression of the intestinal ABCG2 is considered as a new target for treating hyperuricemia and gout. However, at present, no medicine targeting the intestinal ABCG2 has been found.

Probiotics are safe and have no side effects. At present, many clinical and animal experimental studies have shown that probiotics can relieve obesity, non-alcoholic fatty liver diseases, inflammatory bowel diseases and the like. With the continuous in-depth research on probiotics, improving the physical condition of patients with hyperuricemia by means of probiotics has become a new means for treating hyperuricemia and preventing gout.

### SUMMARY

The present disclosure provides Lactobacillus gasseri CCFM1133, which has been preserved in the Guangdong Microbial Culture Collection Center on Jul. 22, 2020 with the preservation number of GDMCC No: 61094.

The L. gasseri CCFM1133 has the following characteristics:
(1) bacterial characteristics: gram stain positive, nonspore and non-motile bacteria;
(2) colony characteristics: offwhite, round, glossy, having a slight fluctuation and an unsmooth edge;
(3) growth characteristics: under anaerobic conditions at constant temperature of 37° C., culturing in an MRS culture medium for about 12 h to arrive at the telophase of a logarithmic phase; and
(4) high tolerance to simulated gastric and intestinal fluid.

The present disclosure further provides a composition including the L. gasseri CCFM1133.

In one embodiment, the quantity of the L. gasseri CCFM1133 is greater than or equal to 1×10⁶ CFU/mL or greater than or equal to 1×10⁶ CFU/g.

In one embodiment, the quantity of the L. gasseri CCFM1133 is greater than or equal to 1×10⁹ CFU/mL or greater than or equal to 1×10⁹ CFU/g.

In one embodiment, the composition includes, but is not limited to, microbial preparations, functional food, health-care products or medicines.

In one embodiment, the composition includes a live strain, a dry strain, a strain metabolite or an inactivated strain of the L. gasseri CCFM1133.

In one embodiment, the composition is a 3% sucrose solution including the L. gasseri CCFM1133.

The present disclosure further provides application of the L. gasseri CCFM1133 in preventing and/or treating and relieving hyperuricemia and gout.

In one embodiment, the application is to administer the L. gasseri CCFM1133 or the composition including the L. gasseri CCFM1133 to a subject.

In one embodiment, the application includes, but is not limited to, at least one of the following effects:
(1) reducing serum uric acid levels of mammals with hyperuricemia;
(2) reducing the activity of serum and liver xanthine oxidase (XOD) of the mammals with hyperuricemia;
(3) reducing glucose levels of the mammals with hyperuricemia;
(4) reducing triglyceride levels of the mammals with hyperuricemia;
(5) promoting the production of intestinal short-chain fatty acids of the mammals with hyperuricemia;
(6) increasing the activity of liver catalase (CAT) and glutathione peroxidase (GSH-Px) of the mammals with hyperuricemia; and
(7) increasing the expression of ileum uric acid transporter ABCG2 of the mammals with hyperuricemia.

In one embodiment, the mammals include, but are not limited to, human.

In one embodiment, the medicine further includes a pharmaceutically acceptable carrier.

In one embodiment, the pharmaceutically acceptable carrier includes, but is not limited to, one or more of a filling agent, a wetting agent, a disintegrating agent, a binding agent or a lubricating agent.

In one embodiment, the filling agent is one or more of microcrystalline cellulose, lactose, mannitol, starch or dextrin; the wetting agent is one or more of ethanol or glycerinum; the disintegrating agent is one or more of sodium carboxymethyl starch, crosslinked sodium carboxymethyl starch, crosslinked povidone or low-substituted hydroxypropyl cellulose; the binding agent is one or more of starch paste, syrup, maltose, refined honey or liquid glucose; and the lubricating agent is one or more of magnesium stearate, sodium stearyl fumarate, talcum powder or silicon dioxide.

The present disclosure further sets forth application of the L. gasseri CCFM1133 in preparing fermented food.

In one embodiment, the application includes, but is not limited to, using the L. gasseri CCFM1133 as a fermentation microorganism to perform fermentation by using food materials.

The present disclosure further provides a method for preventing and/or treating metabolic diseases caused by purine metabolism disturbance, where the method includes ingesting the L. gasseri CCFM1133 or the composition into intestinal tracts.

In one embodiment, the metabolic diseases caused by purine metabolism disturbance include hyperuricemia and/or gout.

In one embodiment, the preventing and/or treating metabolic diseases caused by purine metabolism disturbance includes any of aspects (1) to (7):
(1) reducing serum uric acid levels;
(2) reducing the activity of serum and liver xanthine oxidase (XOD);
(3) reducing glucose levels;
(4) reducing triglyceride levels;
(5) promoting the production of intestinal short-chain fatty acids;
(6) increasing the activity of liver catalase and glutathione peroxidase; and
(7) increasing the expression of ileum uric acid transporter ABCG2.

The present disclosure has the beneficial effects that: the L. gasseri CCFM1133 can reduce serum uric acid levels of mice with hyperuricemia, inhibit the activity of serum and liver xanthine oxidase (XOD) of the mice with hyperuricemia, and reduce the occurrence of gout; the L. gasseri CCFM1133 can reduce the glucose and serum triglyceride (TG) levels of the mice, increase the activity of liver catalase (CAT) and glutathione peroxidase (GSH-Px), and assist in relieving obesity, diabetes, non-alcoholic fatty liver diseases and the like; the L. gasseri CCFM1133 can increase the intestinal short-chain fatty acid levels of the mice to promote the health of the mice; and the L. gasseri CCFM1133 can increase the expression of the ileum uric acid transporter ABCG2, and promote the excretion of intestinal uric acid. The L. gasseri CCFM1133 has a wide application prospect.

Biological Material Preservation

L. gasseri CCFM1133 is classified and named as Lactobacillus gasseri, and has been preserved in the Guangdong Microbial Culture Collection Center on Jul. 22, 2020 with the preservation number of GDMCC No: 61094.

### BRIEF DESCRIPTION OF FIGURES

FIG. 1 shows the colony morphology of L. gasseri CCFM1133.

FIG. 2 shows the effect of L. gasseri CCFM1133 on mice with hyperuricemia.

FIG. 3 shows the effect of L. gasseri CCFM1133 on the activity of serum and liver xanthine oxidase (XOD) of mice with hyperuricemia.

FIG. 4 shows the effect of L. gasseri CCFM1133 on fecal short-chain fatty acids of mice with hyperuricemia.

FIG. 5 shows the effect of L. gasseri CCFM1133 on glucose of mice with hyperuricemia.

FIG. 6 shows the effect of L. gasseri CCFM1133 on total triglyceride (TG) of mice with hyperuricemia.

FIG. 7 shows the effect of L. gasseri CCFM1133 on the activity of liver catalase (CAT) and glutathione peroxidase (GSH-Px) of mice with hyperuricemia.

FIG. 8 shows the effect of L. gasseri CCFM1133 on ileum uric acid transporter ABCG2 of mice with hyperuricemia.

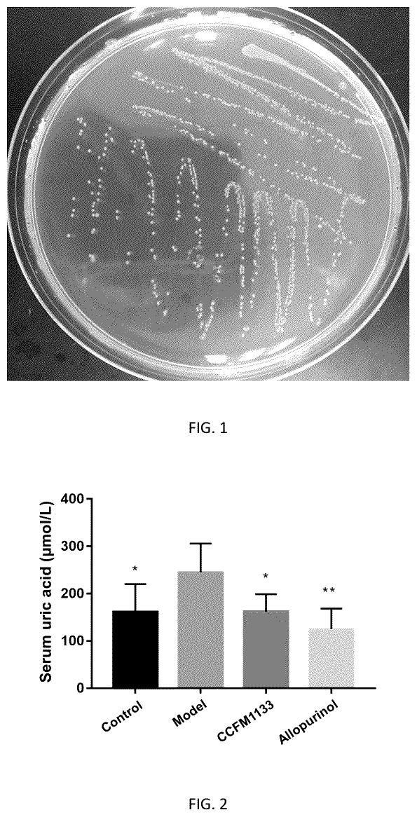

Compared with a hyperuricemia model group, * P<0.05, ** P<0.01, *** P<0.001, **** P<0.0001.

### DETAILED DESCRIPTION

### Example 1 Screening of L. gasseri CCFM1133

(I) Isolation and Screening of Lactobacillus
(1) 1 g of fresh feces was taken from a healthy adult. After gradient dilution, the fresh feces was applied to an LBS culture medium to which 1% nystatin was added. The mixture was put in an incubator at constant temperature of 37° C. to be cultured for 48 h.
(2) After the culture, according to the color, size and edge shapes of colonies, the colonies were selected by using an inoculating ring and striated and purified.
(3) The obtained colonies were subjected to gram staining and catalase analysis.
(4) Gram stain positive bacillus and catalase-negative bacillus were retained.

(II) Molecular Biological Identification of Lactobacillus
(1) Extraction of monobacterial genome
(A) The lactobacillus screened in step (I) was cultured overnight.
(B) 1 mL of bacterial suspension cultured overnight was put in a 1.5 mL centrifuge tube, centrifuged at 10000 r/min for 2 min, and the supernatant was discarded to obtain a thallus.
(C) After the thallus was purged with 1 mL of sterile water, centrifuging was performed at 10000 r/min for 2 min, and the supernatant was discarded to obtain a thallus.
(D) 200 μL of SDS lysis solution was added, and a water bath was performed at 80° C. for 30 min.
(E) 200 μL of phenol-chloroform solution was added to the lysis solution of the thallus, where the phenol-chloroform solution includes Tris-saturated phenol, chloroform and isoamylol at a volume ratio of 25:24:1. After reverse uniform mixing, centrifuging was performed at 12000 rpm for 5 to 10 min, and 200 μL of supernatant was taken.
(F) 400 μL of ice ethanol or ice isopropanol was added to 200 μL of supernatant. Standing was performed at −20° C. for 1 h. Centrifuging was performed at 12000 rpm for 5 to 10 min, and the supernatant was discarded.
(G) 500 μL of 70% (volume percentage) ice ethanol was added to resuspend and precipitate. Centrifuging was performed at 12000 rpm for 1 to 3 min, and the supernatant was discarded. Drying was performed by using an oven at 60° C., or air-drying was performed.
(H) 50 μL of ddH₂O was redissolved to precipitate for PCR.

(2) 16S rDNA PCR
(A) Bacterial 16S rDNA 50 μL PCR reaction system 10×Taq buffer, 5 μL; dNTP, 5 μL; a primer 27F, 0.5 μL; a primer 1492R, 0.5 μL; a Taq enzyme, 0.5 μL; a template, 0.5 μL; ddH₂, 38 μL.
(B) PCR conditions
95° C., 5 min; 95° C., 10 s; 55° C., 30 s; 72° C., 30 s; steps 2-4, 30×; 72° C., 5 min; 12° C., 2 min.
(C) 1% agarose gel was prepared. Then, PCR products were mixed with 10000× loading buffer. The loading amount was 2 μL. The operation was performed at 120 V for 30 min. Then, gel imaging was performed.
(D) The obtained PCR products were sent to a professional sequencing company. The obtained sequencing results were subjected to searching and similarity comparison in the GenBank by using BLAST, and strains identified as L. gasseri were stored at −80° C.

(3) Whole Genome Sequencing

The extracted whole genome was sent to the professional sequencing company. The whole genome of the bacteria was sequenced by using a second-generation sequencer. The obtained sequencing results were subjected to searching and similarity comparison in the GenBank by using BLAST, and a newly discovered strain of the L. gasseri identified according to the sequencing results was stored at −80° C. for later use.

### Example 2: L. gasseri CCFM1133 has Good Tolerance to Simulated Gastric and Intestinal Fluid

The cryopreserved L. gasseri CCFM1133 was inoculated in an MRS culture medium, subjected to anaerobic culture at 37° C. for 14 h, and then subjected to subculturing in an MRS culture solution for 2 to 3 times.

3 mL of culture solution of the L. gasseri CCFM1133 was taken and centrifuged at 8000×g for 2 min to collect thalli. The thalli were mixed with 3 mL of artificially simulated gastric fluid with pH=3.0 (normal saline containing 3 g/L pepsase with pH=3.0), and then was subjected to anaerobic culture at 37° C. Sampling was performed respectively at 0 h and 2 h. Pouring culture was performed in an MRS agar culture medium for plate colony counting. The viable count was measured, and the survival rate was calculated.

3 mL of culture solution of the L. gasseri CCFM1133 was taken and centrifuged at 8000×g for 2 min to collect thalli. The thalli were mixed with 3 mL of artificially simulated intestinal fluid with pH=8.0 (normal saline containing 1 g/L trypsin and 0.3% cholate with pH=8.0), and then was subjected to anaerobic culture at 37° C. Sampling was performed respectively at 0 h, 2 h and 4 h. Pouring culture was performed in an MRS agar culture medium for plate colony counting. The viable count was measured, and the survival rate was calculated.

The survival rate (%) was calculated according to the ratio of the viable count during sampling to the viable count at 0 h in the culture solution. The experimental results are shown in Table 1. The results show that the L. gasseri has good tolerance to the artificially simulated gastric and intestinal fluid.

TABLE 1
Tolerance of L. gasseri CCFM1133 to artificially simulated gastric
and intestinal fluid

| | Artificially | Artificially | |
|---|---|---|---|
| | simulated | simulated intestinal | |
| | gastric fluid | fluid | |
| Processing | 2 | 2 | 4 |
| time (h) | | | |
| Survival rate % | 44.18 ± 7.11 | 59.88 ± 10.16 | 44.61 ± 4.66 |

### Example 3: L. gasseri CCFM1133 has No Toxic or Side Effects on KunMing Mice

The thallus of the L. gasseri CCFM1133 was resuspended in a sucrose solution with the concentration of 30 g/L to prepare a bacterial suspension with the concentration of 4.0×10⁹ CFU/mL. 12 healthy male KunMing mice with the weight of 38 to 44 g were divided into a CCFM1133 group and a control group after a week of acclimatization. 0.3 mL of bacterial suspension with the concentration was given to the CCFM1133 group once a day by intragastric administration. The same amount of 30 g/L sucrose solution without the L. gasseri CCFM1133 was given to the control group by intragastric administration. After a week of observation, death and weight were recorded.

These experimental results are listed in Table 2. These results show that when the L. gasseri CCFM1133 with the concentration of 1×10⁹ CFU/mouse is fed, there is no obvious effect on the mice, there is no significant change in the weight, and no death occurs. The mice have no obvious pathological symptoms.

TABLE 2
Weight change and death of mice

| Time (day) | 1 | 2 | 3 | 4 | 5 | 6 | 7 |
|---|---|---|---|---|---|---|---|
| Weight (g) | 38.33 ± 0.46 | 38.59 ± 0.34 | 39.21 ± 0.38 | 39.54 ± 0.53 | 39.67 ± 0.51 | 39.73 ± 0.55 | 39.98 ± 0.50 |
| of the | | | | | | | |
| CCFM1133 | | | | | | | |
| group | | | | | | | |
| Weight (g) | 38.19 ± 0.37 | 38.87 ± 0.63 | 38.99 ± 0.51 | 39.15 ± 0.72 | 39.39 ± 0.44 | 39.87 ± 0.67 | 40.13 ± 0.52 |
| of the | | | | | | | |
| control | | | | | | | |
| group | | | | | | | |
| Death | — | — | — | — | — | — | — |

### Example 4: L. gasseri CCFM1133 Reduces Serum Uric Acid Levels of Mice with Hyperuricemia

24 healthy male KM mice with the weight of 38 to 44 g were randomly divided into four groups after a week of acclimatization, namely a control group, a hyperuricemia model group, an L. gasseri CCFM1133 intervention group (CCFM1133) and an allopurinol intervention group (allopurinol). Except the control group, 500 mg/kg BW hypoxanthine was given to the other groups every day by intragastric administration, and 100 mg/kg BW oteracil potassium was intraperitoneally injected to the other groups every day. At 1 h before processing with hypoxanthine and oteracil potassium, 0.4 mL of 30 g/L sucrose (a control solvent) was given to the control group and the hyperuricemia model group, L. gasseri CCFM1133 bacterial suspension (30 g/L sucrose solution containing L. gasseri CCFM1133 thallus) with the concentration of 1×10⁹ CFU/mouse was given to the L. gasseri CCFM1133 intervention group, and 5 mg/kg BW allopurinol was given to the allopurinol group. Experimental groups and processing methods are shown in Table 3:

TABLE 3
Groups of experimental animals

| | Daily processing | | Quan- |
|---|---|---|---|
| | methods for 2 to | Daily processing methods | tity of |
| Group | 3 weeks | for 4 to 6 weeks | mice |
| Control group | Control solvent | Control solvent by | 6 |
| | by intragastric | intragastric administration | |
| | administration | Control solvent by | |
| | | intraperitoneal injection | |
| Hyperuricemia | Control solvent | Control solvent by | 6 |
| model group | by intragastric | intragastric administration | |
| | administration | Hypoxanthine by intra- | |
| | | gastric administration | |
| | | Oteracil potassium by | |
| | | intraperitoneal injection | |
| Allopurinol group | 5 mg/kg BW | 5 mg/kg BW allopurinol by | 6 |
| | allopurinol by | intragastric administration | |
| | intragastric | Hypoxanthine by intra- | |
| | administration | gastric administration | |
| | | Oteracil potassium by | |
| | | intraperitoneal injection | |
| L. gasseri | L. gasseri | L. gasseri CCFM1133 with | 6 |
| CCFM1133 | CCFM1133 with | the concentration of 1 × 10⁹ | |
| group | the concentra- | CFU/mouse by intragastric | |
| | tion of | administration | |
| | 1 × 10⁹ CFU/ | Hypoxanthine by intra- | |
| | mouse | gastric administration | |
| | by intragastric | Oteracil potassium by | |
| | administration | intraperitoneal injection | |

At the end of the experiment, fresh feces of the mice was collected and frozen at −80° C. After the experiment was finished, the mice were subjected to fasting but free for drinking water for 12 h. 0.1 mL/10 g of 1% pentobarbital sodium solution was intraperitoneally injected for anesthetization. Then, blood was sampled from eyeballs, and the mice were killed by cervical dislocation. A blood sample was centrifuged at 3500 r/min for min, and the supernatant was taken and frozen at −80° C. for blood index analysis. After liver, ileum and other tissues were taken out, the tissues were quickly rinsed in pre-cooled normal saline to remove blood. The tissues were quick-frozen in liquid nitrogen and transferred for cryopreservation at −80° C. Subsequently, the tissues were prepared into liver homogenate for determining related indexes. The serum uric acid levels were determined by using a kit method.

The effect of the L. gasseri CCFM1133 on the serum uric acid levels of the mice is shown in FIG. 2. Compared with the hyperuricemia model mice, the L. gasseri CCFM1133 can reduce the serum uric acid concentration of the mice with hyperuricemia by 33.67%, which is close to that of the control group; the effect of reducing uric acid is similar to that of the medicine allopurinol; and the occurrence of hyperuricemia and gout can be reduced.

### Example 5: L. gasseri CCFM1133 Reduces the Activity of Xanthine Oxidase of Mice with Hyperuricemia

Experimental animal groups and processing methods are the same as those of Example 4, and xanthine oxidase (XOD) was detected by using a kit method (Beijing Solarbio).

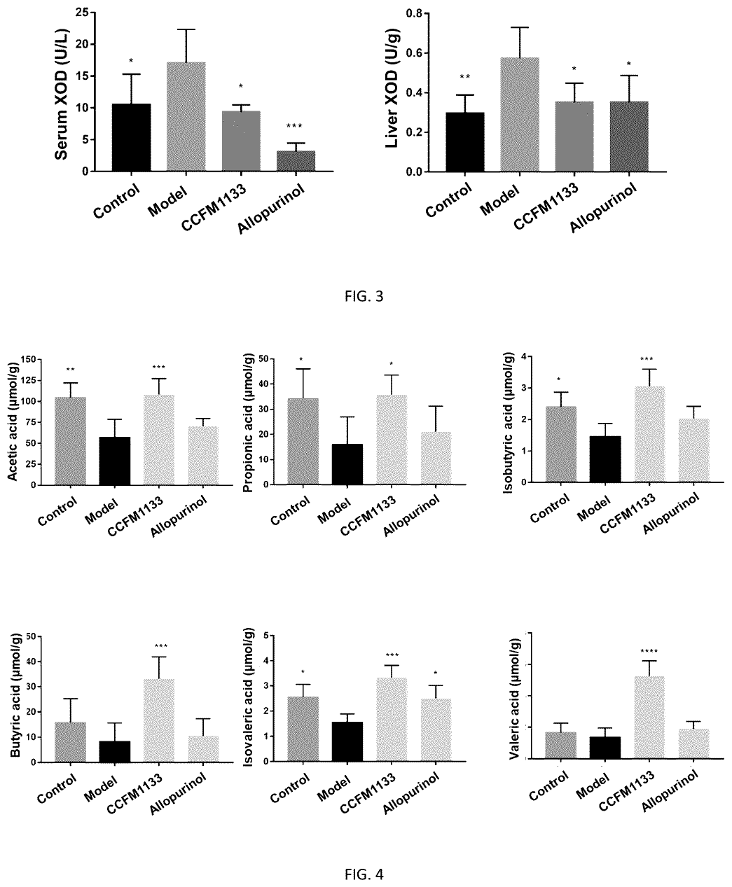

As shown in FIG. 3, compared with the mice with hyperuricemia, the L. gasseri CCFM1133 can reduce the activity of serum and liver xanthine oxidase of the mice with hyperuricemia respectively by 45.30% and 38.84%, so that the increased activity of serum and liver xanthine oxidase of the mice with hyperuricemia tends to be normal. The capability of the L. gasseri CCFM1133 to reduce liver xanthine oxidase is similar to that of the xanthine oxidase inhibiting medicine allopurinol, so that the synthesis of uric acid in the mice is reduced, and the prevention and treatment of hyperuricemia and gout are facilitated.

### Example 6: L. gasseri CCFM1133 Promotes the Production of Intestinal Short-Chain Fatty Acids of Mice

Experimental animal groups and processing methods are the same as those of Example 4. At the end of the experiment, feces of the mice was collected for short-chain fatty acid analysis. The analysis method for the fecal short-chain fatty acids is as follows:

50 mg of fecal sample was weighed. 500 μL of saturated NaCl solution was added and homogenized till no obvious lump existed. After homogenization, 40 μL of 10% sulfuric acid was added for acidification. 1 mL of diethyl ether was added to extract short-chain fatty acids. After oscillation, centrifuging was performed at 12000 r/min and 4° C. for 15 min. After centrifugation, the supernatant was taken and added to a centrifuge tube filled with 0.25 g of anhydrous sodium sulfate for standing. Centrifuging was performed again under the same conditions. After centrifugation, liquid was added to a gas-phase vial for computer analysis.

The short-chain fatty acids were separated by using an Rtx-Wax column. The short-chain fatty acids (acetic acid, propionic acid, isobutyric acid, butyric acid, isovaleric acid and valeric acid) were detected in a full scanning mode (the mass-to-charge ratio scanning range is 33 to 110). Characteristic ions of analyte standards were selected for quantitative analysis.

The results show that (FIG. 4) the fecal short-chain fatty acid (including acetic acid, propionic acid, isobutyric acid, butyric acid, isovaleric acid and valeric acid) levels of the mice with hyperuricemia are lower than that of normal mice, which indicates that hyperuricemia changes intestinal microbial metabolites of the mice, intestinal microorganisms produce less short-chain fatty acids, but the L. gasseri CCFM1133 can reverse the change and promote the production of intestinal short-chain fatty acids. However, allopurinol does not have much of the effect.

### Example 7: L. gasseri CCFM1133 Relieves Glucose Elevation Caused by Hyperuricemia

Experimental animal groups and processing methods are the same as those of Example 4, and glucose was detected by using a Mindray BS480 biochemical analyzer according to a kit method.

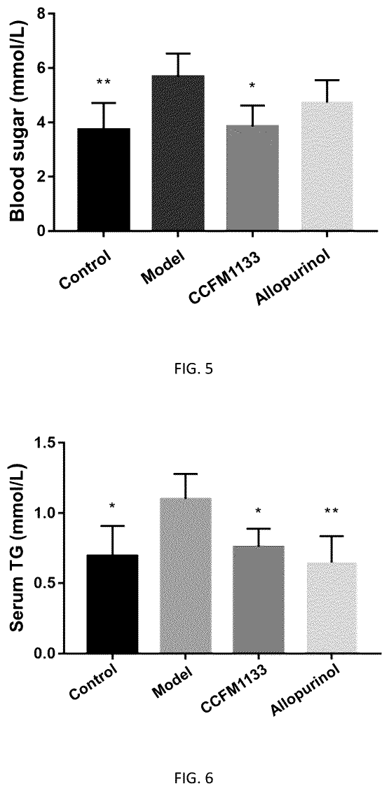

Many studies have shown that diabetes and hyperuricemia as metabolic diseases are related in various ways. The decrease of kidney function caused by chronic diabetes will lead to elevated serum uric acid, which causes hyperuricemia and even gout, and the occurrence of hyperuricemia also increases the risk of diabetes. The glucose results show that (FIG. 5) the glucose concentration of the mice with hyperuricemia reaches 5.67±0.85 mmol/L, but the L. gasseri CCFM1133 can reduce the glucose of the mice with hyperuricemia to be normal, and the effect is better than that of allopurinol, which indicates that the L. gasseri CCFM1133 has the potential to relieve metabolic diseases such as hyperuricemia and diabetes.

### Example 8: L. gasseri CCFM1133 Relieves Total Triglyceride Elevation Caused by Hyperuricemia

Experimental animal groups and processing methods are the same as those of Example 4, and serum total triglyceride (TG) was detected by using a Mindray BS480 biochemical analyzer according to a kit method.

The effect of the L. gasseri CCFM1133 on the serum total triglyceride of the mice with hyperuricemia is shown in FIG. 6. Compared with the control group, the mice with hyperuricemia have a higher serum total triglyceride concentration up to 1.10±0.18 mmol/L. The L. gasseri CCFM1133 can recover the serum total triglyceride concentration to the normal level of 0.76±0.13 mmol/L, and the capability thereof to recover the serum total triglyceride is similar to that of the medicine allopurinol.

### Example 9: L. gasseri CCFM1133 Relieves the Reduction of the Activity of Liver Catalase (CAT) and Glutathione Peroxidase (GSH-Px) Caused by Hyperuricemia

Experimental animal groups and processing methods are the same as those of Example 4, and the activity of liver catalase (CAT) and glutathione peroxidase (GSH-Px) was determined according to a kit method.

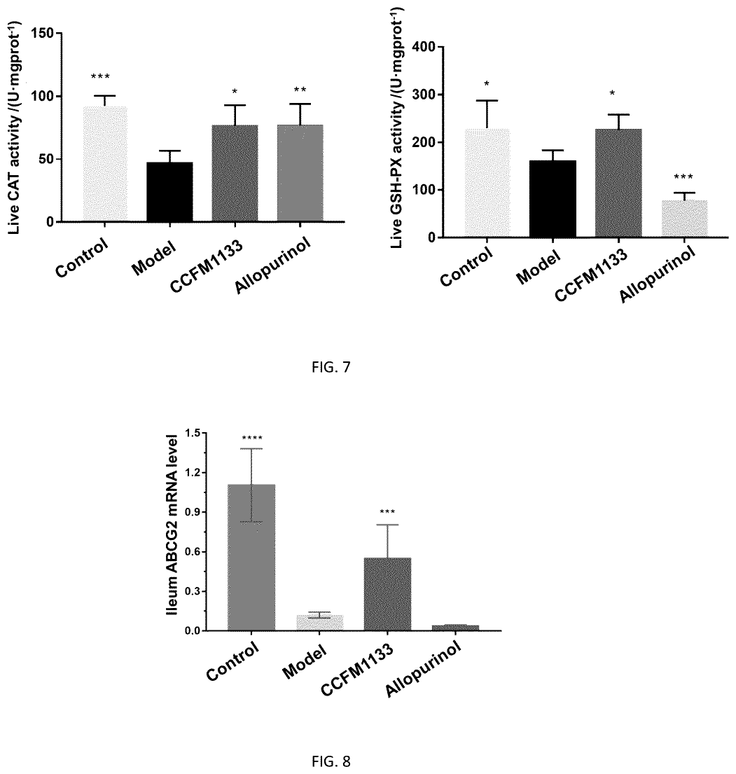

The results show that (FIG. 7) compared with the control group, the mice with hyperuricemia have lower activity of liver catalase and glutathione reductase, which indicates that hyperuricemia makes the liver oxidative stress resistant capability of the mice decrease. The intervention of the L. gasseri CCFM1133 can relieve the reduction of the activity of liver catalase and glutathione reductase caused by hyperuricemia.

### Example 10: L. gasseri CCFM1133 Promotes the Expression of Ileum Uric Acid Transporter ABCG2

Experimental animal groups and processing methods are the same as those of Example 4.

Determination of ileum ABCG2 mRNA: about 20 mg of ileum tissues were added to 500 μL of Trizol, and after ice bathing and homogenization, RNA in the ileum tissues was extracted by using a conventional method. cDNA synthesis was performed according to the instructions of a reverse transcription kit. Samples were mixed with fluorescent dyes SYBR Green super mix (Qiagen, Germany). The PCR system included 5 μL of mix, 1 μL of cDNA and 1 μL of forward and reverse primers, and ddH₂O was supplemented to a total volume of 10 μL. Detection was performed on a real-time fluorescence quantitative gene amplification instrument CFX96™ Real-Time System (Bio-Rad, USA). Each sample was provided with 3 parallel holes, and GAPDH was used as an internal reference. The obtained results were analyzed by using a 2^{-ΔΔCq} method. The used primer sequence is shown in Table 4.

TABLE 4
qPCR primer sequence

| Gene | Sequence | |
|---|---|---|
| GAPDH | F-5′-TCCTGCACCACCAACTGCT | SEQ ID NO. 1 |
| | R-5′-GTCAGATCCACGACGGACACA | SEQ ID NO. 2 |
| ABCG2 | F-5′-TGCCAGATAAGAGGGGTTAGGT | SEQ ID NO. 3 |
| | R-5′-TGCTTGCAGTGGAGTTGAGA | SEQ ID NO. 4 |

The results show that (FIG. 8) the L. gasseri CCFM1133 can obviously increase the mRNA level of the ileum ABCG2 of the mice with hyperuricemia. The ileum ABCG2 plays an important role in the excretion of intestinal uric acid, and the L. gasseri CCFM1133 can promote the excretion of uric acid by increasing the expression of the ileum ABCG2.

### Comparative Example 1

The specific implementation mode is the same as that of Example 4, with the difference lies in that the L. gasseri CCFM1133 is replaced with L. gasseri FHeNJZ11L9 (reported in: Zhou Xingya. Screening, genome comparison and safety assessment of Lactobacillus gasseri and Lactobacillus paragasseri [D]. Jiangnan University, 2019), and the serum uric acid indexes of the mice are determined. The results show that the serum uric acid level of the mice in the L. gasseri FHeNJZ11L9 group is 215.7±39.8 μmol/L, and compared with the hyperuricemia model group (244.7±61.0 μmol/L), the uric acid level of the mice with hyperuricemia has no obvious change.

### Comparative Example 2

The specific implementation mode is the same as that of Example 5, with the difference lies in that the L. gasseri CCFM1133 is replaced with L. gasseri FHeNJZ11L9, and the activity of serum and liver xanthine oxidase of the mice is determined. The results show that the activity of serum and liver xanthine oxidase of the mice in the L. gasseri FHeNJZ11L9 group is 13.355±6.990 U/L and 0.476±0.089 U/g respectively, and compared with the hyperuricemia model group (17.061±5.269 U/L and 0.573±0.157 U/g), the L. gasseri FHeNJZ11L9 reduces the activity of serum and liver xanthine oxidase of the mice with hyperuricemia by 21.72% and 16.93% respectively.

Although the present disclosure has been disclosed with reference to the exemplary examples, they are not intended to limit the present disclosure. Any person skilled in the art can make various changes and modifications without departing from the spirit and scope of the present disclosure, therefore the scope of protection of the present disclosure shall be subject to the scope defined by the claims.

What is claimed is:

1. A composition comprising Lactobacillus gasseri CCFM1133, having been preserved in the Guangdong Microbial Culture Collection Center on Jul. 22, 2020 with the preservation number of GDMCC No: 61094.

2. The composition according to claim 1, wherein the quantity of the L. gasseri CCFM1133 is greater than or equal to 1×10⁶ CFU/mL or greater than or equal to 1×10⁶ CFU/g.

3. The composition according to claim 2, wherein the composition comprises a live strain, a dry strain, a strain metabolite or an inactivated strain of the L. gasseri CCFM1133.

4. The composition according to claim 3, wherein the composition comprises microbial preparations, functional food, health-care products or medicines.

5. The composition according to claim 4, wherein the medicine further comprises a pharmaceutically acceptable carrier.

6. A method for preventing and/or treating and relieving hyperuricemia and gout, comprising administering the composition according to claim 1 to a subject.

7. The method according to claim 6, wherein the preventing and/or treating and relieving hyperuricemia and gout comprises at least one of the following effects:
(1) reducing serum uric acid levels;
(2) reducing the activity of serum and liver xanthine oxidase (XOD);
(3) reducing glucose levels;
(4) reducing triglyceride levels;
(5) promoting the production of intestinal short-chain fatty acids;
(6) increasing the activity of liver catalase and glutathione peroxidase; and
(7) increasing the expression of ileum uric acid transporter ABCG2.

8. The method according to claim 7, wherein the subject comprises human.

9. A method for preventing and/or treating metabolic diseases caused by purine metabolism disturbance, wherein L. gasseri CCFM1133 or the composition according to claim 1 is ingested into intestinal tracts.

10. The method according to claim 9, wherein the metabolic diseases caused by purine metabolism disturbance comprise hyperuricemia and/or gout.

11. The method according to claim 10, wherein the preventing and/or treating metabolic diseases caused by purine metabolism disturbance comprises any one of aspects (1) to (7):
(1) reducing serum uric acid levels;
(2) reducing the activity of serum and liver xanthine oxidase (XOD);
(3) reducing glucose levels;
(4) reducing triglyceride levels;
(5) promoting the production of intestinal short-chain fatty acids;
(6) increasing the activity of liver catalase and glutathione peroxidase; and
(7) increasing the expression of ileum uric acid transporter ABCG2.


SEQUENCE LISTING (4 sequences)

SEQ ID NO: 1 - DNA, 19 bp, synthetic construct
  source 1..19: mol_type = other DNA
tcctgcaccaccaactgct

SEQ ID NO: 2 - DNA, 21 bp, synthetic construct
  source 1..21: mol_type = other DNA
gtcagatccacgacggacaca

SEQ ID NO: 3 - DNA, 22 bp, synthetic construct
  source 1..22: mol_type = other DNA
tgccagataagaggggttaggt

SEQ ID NO: 4 - DNA, 20 bp, synthetic construct
  source 1..20: mol_type = other DNA
tgcttgcagtggagttgaga